(19) United States
(12) Patent Application Publication
(10) Pub. No.: US 2019/0040197 A1
(43) Pub. Date: Feb. 7, 2019

(54) Modifying agent, polyamide copolymer synthesized using modifying agent and method for preparing polyamide copolymer

(71) Applicant: National Taipei University of Technology, Taipei (TW)

(72) Inventors: SYANG-PENG RWEI, Taipei (TW); Tun-Fun Way, Taipei (TW); Yu-Hao Chen, Taipei (TW)

(73) Assignee: National Taipei University of Technology, Taipei (TW)

(21) Appl. No.: 16/045,747

(22) Filed: Jul. 26, 2018

Related U.S. Application Data
(60) Provisional application No. 62/539,547, filed on Aug. 1, 2017.

(51) Int. Cl. C08G 69/16; C07C 233/36

(52) U.S. Cl. CPC C08G 69/16; C07C 233/36

(57) ABSTRACT

The invention provides a modifying agent, for synthesizing a polyamine copolymer, being a amide- group-containing diamine which is a reaction product of an aliphatic diamine and a first carboxylate, or a salt formed by reacting the amide- group-containing diamine with a second carboxylic acid, wherein the amide- group-containing diamine is represented by the general formula (I) where y represents an integer within 2˜6 (2 ≤y≤6) and x represents an integer within 4˜18 (4≤x≤18); the first carboxylate is an ester formed from an aliphatic carboxylic acid and the second carboxylic acid is a carboxylic acid.

### BACKGROUND OF THE INVENTION

### a. Field of the Invention

The invention relates to a novel modifying agent for nylon preparation, a modified polyamide copolymer using the same and method for preparing the same, particularly to a novel modifying agent for nylon preparation and a modified polyamide for hot-melt adhesive and low melt nylon copolymers.

### b. Description of the Related Art

Polyamide (PA), commonly known as nylon, is a polymer containing a functional group of an amide group (—CO—NH—) in the main chain. Since the abundant hydrogen bonds between the polymer chains are attracted to each other, they have excellent properties such as mechanical strength, heat resistance, abrasion resistance, chemical resistance, and hygroscopicity. The use of PA6 and/or PA66 in nylon fiber accounts for 90% of the total products. Its main raw materials are ϵ-caprolactam, adipic acid and hexamethylene diamine, and it is also the main raw material of other nylon series products. Therefore, the production of raw materials plays a very important role in its future development.

Furthermore, the low melting point nylon fiber can be used as a hot melt adhesive, and the fiber as a thermal filament can be applied to a woven fabric, a fly-knit shoe upper, a hosiery, a hot melt bonding, etc. The polymers having two different melting points are utilized and subjected to thermal processing to shape and bond the woven fabric to achieve the better fabric quality. As the thermal filaments are bonded, they are superior to the use of ordinary glue. The advantage is that it can be processed by thermal treatment after sewing and the fabrication process does not require the use of solvent and is a time saving process, thereby shortening the process and reducing the production cost. The process is economical and environmentally friendly.

Monomers used in the synthesis of nylon, such as hexamethylenediamine (HMDA), are the most widely produced diamines in the world, 99% of which are used in the production of PA66 and its series products. The development of PA66 in Asia is inferior to that in Europe and America. The main reason is that the production of hexamethylenediamine is limited to its raw material adiponitrile. At present, the advanced production technology of adiponitrile in the world is monopolized by only a few manufacturers.

Therefore, the modification of the polyamide fiber product and the diversification of the monomeric raw materials is essential to provide a new structure, performance and application areas of the polyamide fiber so as to achieve the functionalization of the product to give the high added value and expand the market for the polyamide fiber.

### BRIEF SUMMARY OF THE INVENTION

In light of the above background, in order to fulfill the requirements of the industry, one object of the invention provides a modifying agent, in particular, a modifying agent for modifying nylon properties. The modifying agent is a diamine obtained by reacting a diamine having a low carbon number with a dicarboxylic acid. The process does not need to use high-cost hexamethylenediamine and is simple compared to the traditional nylon fabrication process to reduce the manufacturing cost. In addition, the characteristics of the polyamide copolymer can be varied, depending on the modifying agent to be used. On the other hand, the salt obtained by reacting the modifying agent with the dicarboxylic acid (which can be different from the dicarboxylic acid used in the reaction of preparing the modifying agent) may be used. When the polyamide is synthesized, the reaction can be carried out under normal atmospheric pressure (for example 1 atm) without having to be carried out under a high pressure environment. In addition to the advantages of the easy reaction conditions, the hygroscopicity (absorption of moisture) of the nylon fiber can be improved, the cool-feeling is better, and the T-peel strength can be improved when used as a hot melt adhesive.

Other objects and advantages of the invention can be better understood from the technical characteristics disclosed by the invention. In order to achieve one of the above purposes, all the purposes, or other purposes, one embodiment of the invention provides a modifying agent, for synthesizing a polyamine copolymer, being a amide- group-containing diamine which is a reaction product of an aliphatic diamine and a first carboxylate, or a salt formed by reaction of the amide- group-containing diamine with a second carboxylic acid, wherein the amide- group-containing diamine is represented by the following general formula (I):

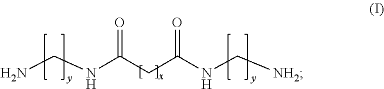

In the general formula (I), y represents an integer within 2˜6 (2≤y≤6) and x represents an integer within 4˜18 (4≤x≤18); the first carboxylate is an ester formed from an aliphatic carboxylic acid and the second carboxylic acid is a carboxylic acid.

Furthermore, one other embodiment of the invention provides a method for preparing a polyamide copolymer, comprising the following steps: a step of providing a modifying agent, being a amide- group-containing diamine which is a reaction product of an aliphatic diamine and a first carboxylate, or a salt formed by reaction of the amide- group-containing diamine with a second carboxylic acid; and a step of copolymerizing the modifying agent with caprolactam; wherein the amide- group-containing diamine is represented by the general formula (I), where y represents an integer within 2˜6 (2≤y≤6) and x represents an integer within 4˜18 (4≤x≤18); the first carboxylate is an ester formed from an aliphatic carboxylic acid and the second carboxylic acid is a carboxylic acid.

One other embodiment of the invention provides a polyamide copolymer which is obtained from the reaction of the above modifying agent with caprolactam or the method for preparing a polyamide copolymer according to the present invention.

According to the present invention, a modifying agent, a polyamide copolymer synthesized by reacting the modifying agent with caprolactam, and the method for preparing a polyamide copolymer are provided. The obtained polyamide copolymer has a low melting temperature and higher moisture regain than the traditional nylon 6 homopolymer. In addition, the obtained polyamide copolymer has a high T-peel strength suitable to be used as a low-melt adhesive. On the other hand, the method for preparing a polyamide copolymer according to the present invention has the advantages of using low cost raw materials and having easy processing conditions.

Other objectives, features and advantages of the invention will be further understood from the further technological features disclosed by the embodiments of the invention wherein there are shown and described preferred embodiments of this invention, simply by way of illustration of modes best suitable to carry out the invention.

### BRIEF DESCRIPTION OF THE DRAWINGS

FIG. 1 shows H-NMR and FTIR spectra and their analysis of the modifying agent (amide- group-containing diamine: BAEA/AA salt) according to one embodiment of the invention.

FIG. 2 shows the thermal analysis of the modifying agent (amide- group-containing diamine: BAEA) according to one embodiment of the invention.

FIG. 3 shows H-NMR and FTIR spectra and their analysis of the modifying agent (product of BAEA and adipic acid, BAEA/AA salt) according to another embodiment of the invention.

FIG. 4 shows the thermal analysis of the modifying agent (BAEA/AA salt) according to another embodiment of the invention.

FIG. 5 shows FTIR spectra their analysis of the polyamide copolymers of examples 1 and 2 and the reference example 1 according to one embodiment of the invention.

FIG. 6 shows DSC results of the polyamide copolymers of examples 1 and 2 and the reference example 1 according to one embodiment of the invention.

FIG. 7 shows DMA results and glass transition temperatures of the polyamide copolymers of examples 1 and 2 and the reference example 1 according to one embodiment of the invention.

FIG. 8 shows H-NMR spectrum of the modifying agent (BAES) according to another embodiment of the invention.

FIG. 9 shows H-NMR spectrum of the modifying agent (BAHA) according to another embodiment of the invention.

FIG. 10 shows H-NMR spectrum of the modifying agent (BAHA/IPA salt) according to another embodiment of the invention.

### DETAILED DESCRIPTION OF THE INVENTION

In the following detailed description of the preferred embodiments, reference is made to the accompanying drawings which form a part hereof, and in which are shown by way of illustration specific embodiments in which the invention may be practiced. Some preferred embodiments of the present invention will now be described in greater detail in the following. Detail descriptions of the structure and elements will be provided in the following in order to make the invention thoroughly understood. Obviously, the application of the invention is not confined to specific details familiar to those who are skilled in the art. On the other hand, the common structures and elements that are known to everyone are not described in details to avoid unnecessary limits of the invention. Some preferred embodiments of the present invention will now be described in greater detail in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

According to one embodiment of the invention, a modifying agent is disclosed. The modifying agent is for synthesizing a polyamine copolymer, and is an amide- group-containing diamine or a diamine dicarboxylate salt. The amide- group-containing diamine is a reaction product of an aliphatic diamine and a first carboxylate. The diamine dicarboxylate salt is formed by reacting the amide- group-containing diamine with a second carboxylic acid. The amide- group-containing diamine is reprecented by the following general formula (I):

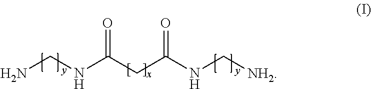

In the general formula (I), y represents an integer within 2˜6 (2≤y≤6) and x represents an integer within 4˜18 (4≤x≤18); the first carboxylate is an ester formed from an aliphatic carboxylic acid and the second carboxylic acid is a carboxylic acid.

Specifically, the aliphatic diamine is, for example, ethylenediamine, propylenediamine, butanediamine, pentanediamine, hexamethylenediamine or the like. More preferably, the aliphatic diamine is ethylenediamine, propylenediamine or butanediamine and more ideally being ethylenediamine. The first carboxylate, for example, can be adipate, suberate, sebacate, dodecanedioate, tetradecanedioate, hexadecandioate, octadecandioate. Preferably, it is an adipate, a suberate, a sebacate or a dodecanedioate and more preferably an adipate, a sebacate or a dodecanedioate. The second carboxylic acid is, for example, isophthalic acid, terephthalic acid, adipic acid, suberic acid, sebacic acid, dodecanedioic acid, tetradecanedioic acid, hexadecandioic acid, octadecanic acid, and their corresponding isomers, etc.

The amide- group-containing diamine has a boiling point higher than 180° C., more preferably a boiling point of 200° C. or higher. A diamine formed by reacting a diamine having a low carbon number (i.e. including only a small number of carbons in a molecule) and a low boiling point with a dicarboxylate (the first dicarboxylate) can have a boiling point higher than that of the diamine of the reactant. Thus, the copolymerization using the diamine (amide- group-containing diamine) can be carried out under the normal pressure, such as 1 atm, without using a special reactor.

Furthermore, in one embodiment, the modifying agent is a diamine dicarboxylate salt which is a reaction product of the amide- group-containing diamine and the second carboxylic acid selected from the group consisting of the following or the combination thereof: isophthalic acid, terephthalic acid and adipic acid.

According to another embodiment of the present invention, a polyamide copolymer is disclosed. The polyamide copolymer is synthesized by reacting one of the modifying agents according to the present invention with caprolactam, wherein the polyamide copolymer has a lower melting temperature than polycaprolactam homopolymer (PA6 or nylon 6).

In one embodiment, the polyamide copolymer ccording to the present invention has a moisture regain more than 5% at 20° C. and 65% RH (relative humidity).

According to another embodiment of the present invention, a method for preparing a polyamide copolymer is disclosed. The method comprises the following steps: a step of providing a modifying agent, being a amide- group-containing diamine which is a reaction product of an aliphatic diamine and a first carboxylate (amination of an ester), or a salt formed by reaction of the amide- group-containing diamine with a second carboxylic acid; and a step of copolymerizing the modifying agent with caprolactam; wherein the amide- group-containing diamine is represented by the general formula (I).

In one embodiment, the step of copolymerizing is conducted under an atmospheric pressure of 1 atm.

The examples of the aliphatic diamine, the first carboxylate, and the second carboxylic acid are the same as those shown in the above paragraphs.

In the following, the specific examples are shown to further illustrate the present invention but the scope of the present invention is not limited to the following examples.

### EXAMPLE 1

(Synthesis of Amide- Group-Containing Diamine; BAEA)

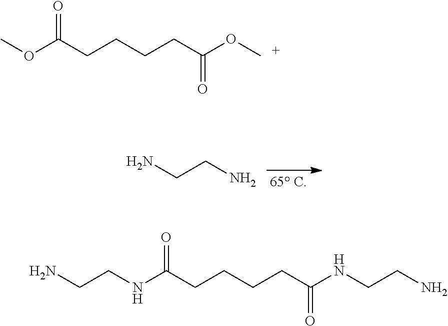

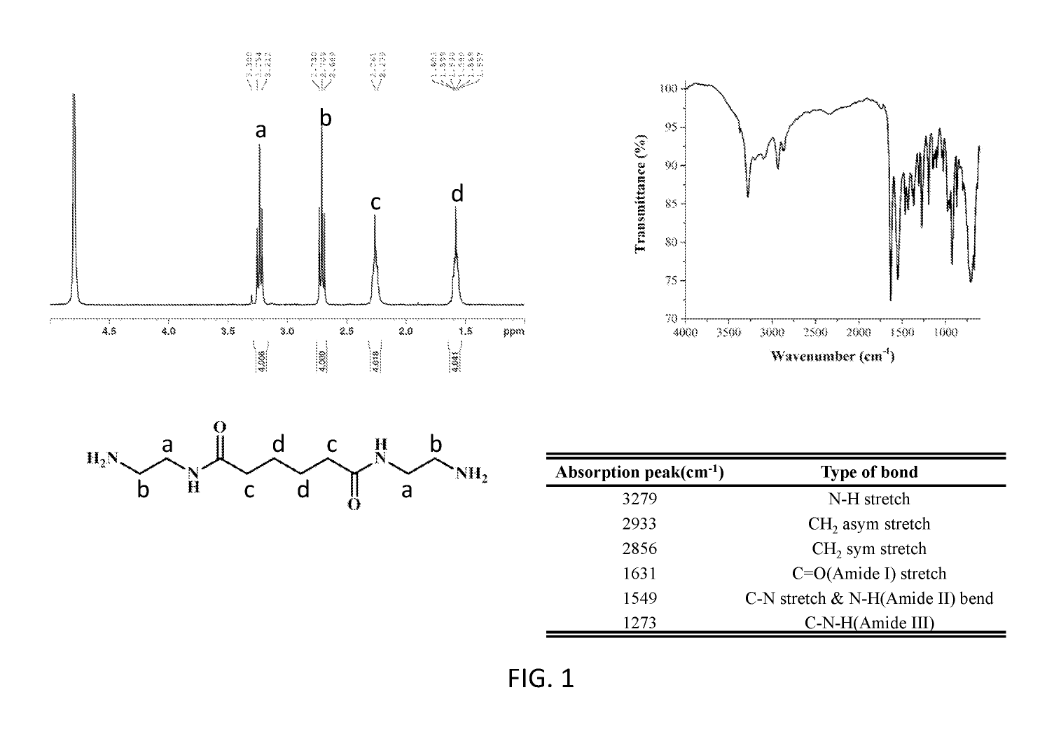

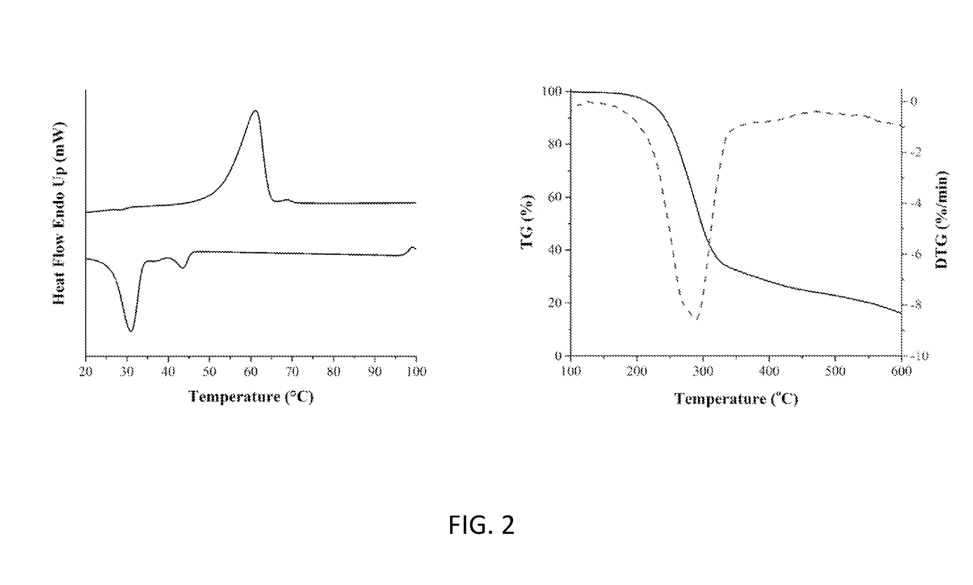

Dimethyl adipate (159.5 g, 0.92 mole, Dimethyl adipate, DMA) and ethylenediamine (550.2 g, 9.16 mole, Ethylenediamide, EDA) were weighed and added to a 1 L reaction flask, reacted at 65° C. under nitrogen atmosphere for 6.0 h. After standing until cooled, the white solid oligomer was obtained via filtration and then thoroughly rinsed with ethanol. The lower filtrate was collected and distilled under the reduced pressure at 80° C. to obtain white solids. The white solids were dried in an oven at 50° C. for one day to obtain white powder solids (Yelid=66.5-75.2%). FIG. 1 shows H-NMR and FTIR spectra and their analysis of the modifying agent (amide- group-containing diamine: BAEA) according to one embodiment of the invention. FIG. 2 shows the thermal analysis of the modifying agent (amide- group-containing diamine: BAEA) according to one embodiment of the invention.

(Synthesis of BAEA/AA Salt)

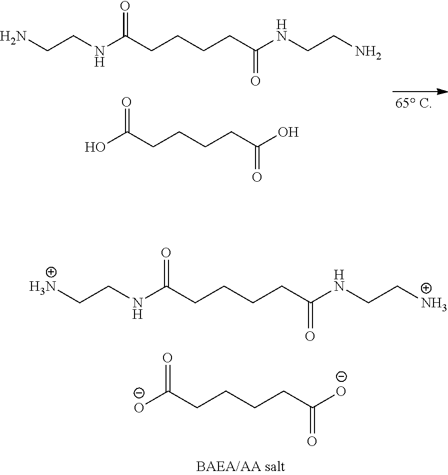 [SMILES: NCCNC(=O)CCCCC(=O)NCCN ; O=C(O)CCCCC(=O)O ; [NH3+]CCNC(=O)CCCCC(=O)NCC[NH3+] ; O=C([O-])CCCCC(=O)[O-]]

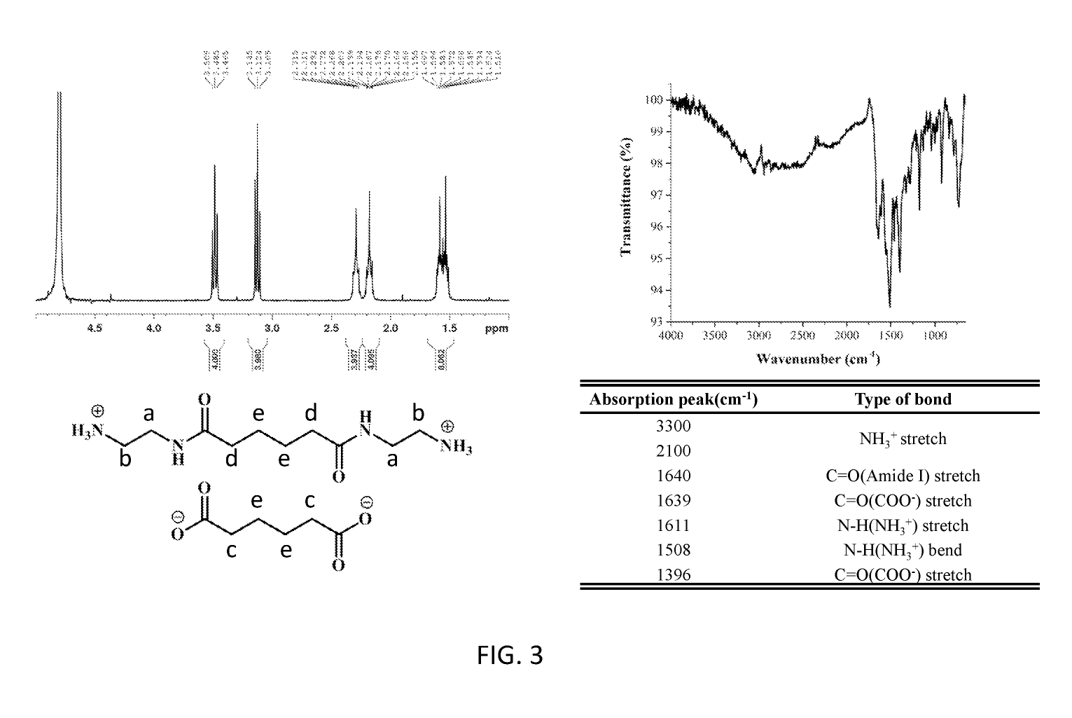

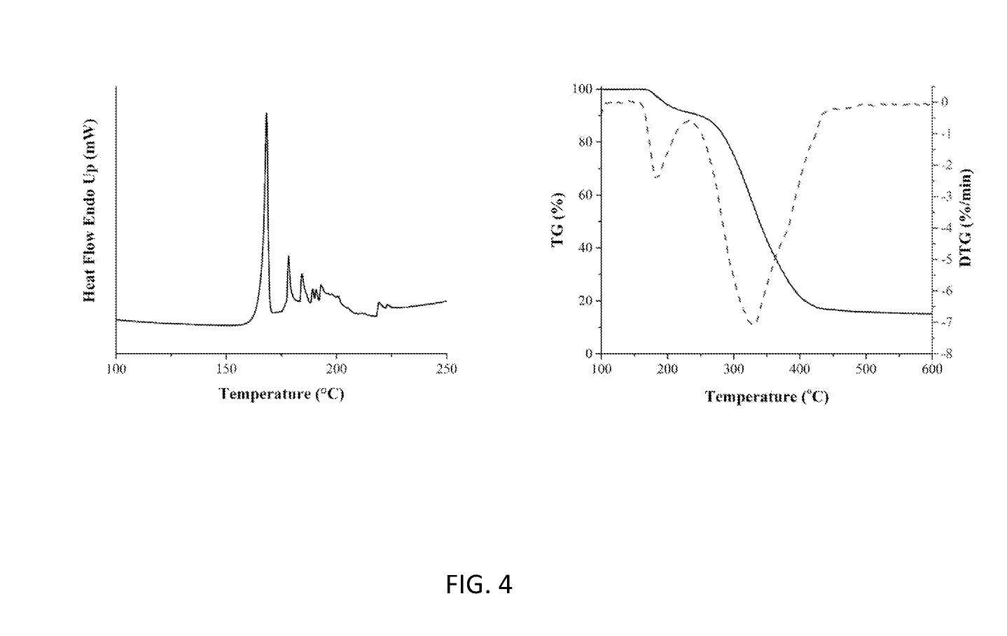

A solution of BAEA (125.0 g, 0.54 mole) in ethanol and a solution of adipic acid (79.3 g, 0.54 mole, Adipic acid, AA) in ethanol were prepared and placed in two beakers and dissolved by heating to 65° C. The ethanol solution of adipic acid was slowly poured into a solution of BAEA in ethanol, and the reaction was taken place while the mixture solution was stirred for 6.0 h. The crystals were precipitated by cooling to room temperature, and the white crystalline solids were filtered and thoroughly rinsed with ethanol. The solids were collected and dried in a vacuum oven at 80° C. for one day to obtain BAEA/AA salt (white powdery salt) (Yelid=93.0-96.5%). FIG. 3 shows H-NMR and FTIR spectra and their analysis of the modifying agent (product of BAEA and adipic acid, BAEA/AA salt) according to another embodiment of the invention. FIG. 4 shows the thermal analysis of the modifying agent (BAEA/AA salt) according to another embodiment of the invention.

(Synthesis of Polyamide Copolymer)

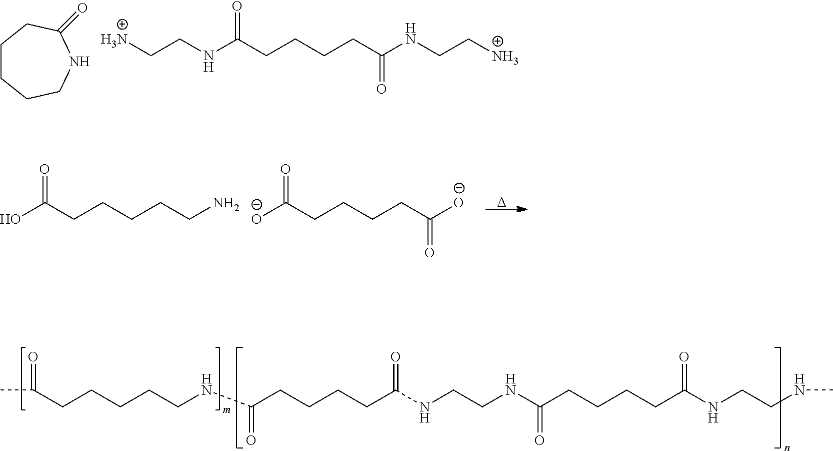

ϵ-Caprolactam (299.0 g, 2.64 mole, ϵ-Caprolactam, ϵ-CPL) was added to a 2 L-reactor at room temperature, and 5.0 wt % of aminocaproic acid (15.0 g, 0.11 mole) was added thereto. Aminocaproic acid (ACS), deionized water (100 g, 5.55 mole) and antioxidant 1098 (0.4 g, 0.1 wt %) (CHINOX 1098, available from Double Bond Chemical) were added to the reactor. The stirring rate was set to 52 rpm. After the reactor was vacuumed three times and filled with nitrogen, the temperature of the reactor was set to 200° C. and the temperature was gradually raised. The reaction was taken place for 2.0 h. After the prepolymerization of PA6, BAEA/AA salt (86.0 g, 0.23 mole), NaH₂PO₂ (0.4 g, 0.1%) and deionized water (100 g, 5.55 mole) were added and the reactor were kept at 200° C. to react for 2.0 h. The temperature of the reactor was gradually raised to 250° C. After 12 to 16 h, the viscosity of the copolymer was increased under the vacuum environment (about 1 torr). When the torque value (Torque) is increased by 40%, the reaction was stopped and the product was cut to pellets.

T-Peel Strength Test (T-Peel) Test

According to the specification of ASTM D1876-01, the prepared polymer was placed between the two stripes of nylon fabrics (317×25 mm) to obtain a test laminate sample. The test sample was processed by hot press at a condition of 190° C. and 10 kg/cm² as the peeling test sample. The peeling test was conducted at a peeling rate of 254 mm/min and the peel strength was taken as the average value of the peak value and the valley value.

Melt Spinning

The raw materials were dried and placed in a feeder, and the appropriate screw temperature, die temperature, feed rate, screw speed, and coiling rate are adjusted, depending on the nature of the raw material. The melt-spinning experiment used a 2.0 mm single-hole spinneret with a screw temperature of about Tm+50° C.; a die temperature of Tm+20° C.; a feed rate of 5 rpm; and a screw speed of 30 rpm. The melt spinning of the test raw material was conducted and fibers were then obtained.

Moisture Regain of Fibers

The moisture regain of a specimen is defined as: the equilibrium weight of water contained by a specimen expressed as a percentage of its oven-dry weight. A small bundle of the melt-spun fibers was cut and placed in a vacuum oven to dry at 100° C. for 24 h and the weight was recorded to be W₀. The fiber sample was placed in a constant temperature and humidity chamber according to ASTM-D1776-04. The humidity of the test instrument is set to 65% and the temperature is set to 20° C. After 72 h, the fiber sample was taken out and the weight was recorded to be W₁. The moisture regain of the fiber sample was obtained using the following equation:

Moisture regain (%)=[(W₁−W₀)/W₀]/×100.

### EXAMPLE 2

Except the quantities of the raw materials which were shown in Table 1, the polyamide copolymer of example 2 was prepared using the same method as described in example 1.

TABLE 1
The feeding ratios of the components to
obtain PA6 and polyamide copolymers

| | | | | | | | anti- |
|---|---|---|---|---|---|---|---|
| | | | | | | | oxidant |
| | PA6 (mole) | | | PA-salt (mole) | | catalyst | (g) |
| | | | DI- | BAEA/ | DI- | (g) | CHINOX |
| | CPL | ACS | water | AA salt | water | NaH₂PO₂ | 1098 |
| Reference | 3.36 | 0.15 | 5.5 | — | — | 0.4 | 0.4 |
| (100.0/ | | | | | | | |
| 0.0) | | | | | | | |
| Example 1 | 2.64 | 0.11 | 5.5 | 0.23 | 5.5 | 0.4 | 0.4 |
| (92.4/7.6) | | | | | | | |
| Example 2 | 2.45 | 0.11 | 5.5 | 0.29 | 5.5 | 0.4 | 0.4 |
| (90.0/ | | | | | | | |
| 10.0) | | | | | | | |

“—” indicates no addition.

### REFERENCE EXAMPLE

Except the quantities of the raw materials which were shown in Table 1, the polyamide copolymer of reference example was prepared using the same method as described in example 1.

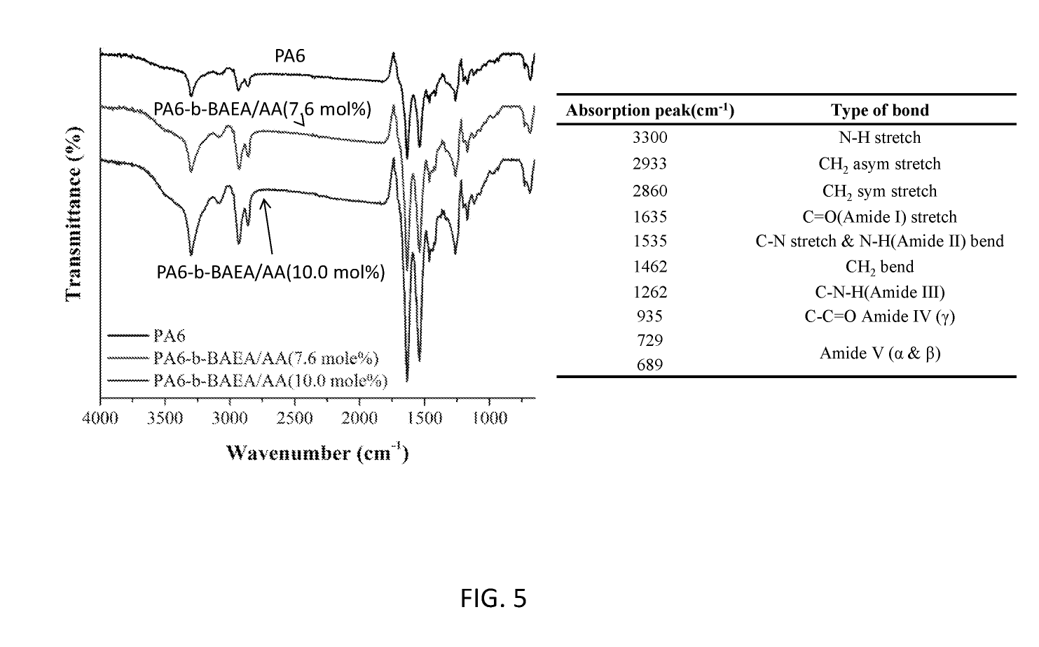

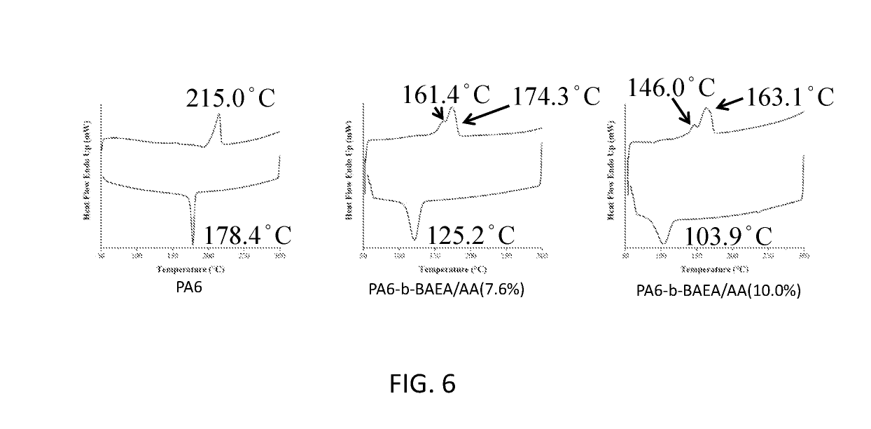

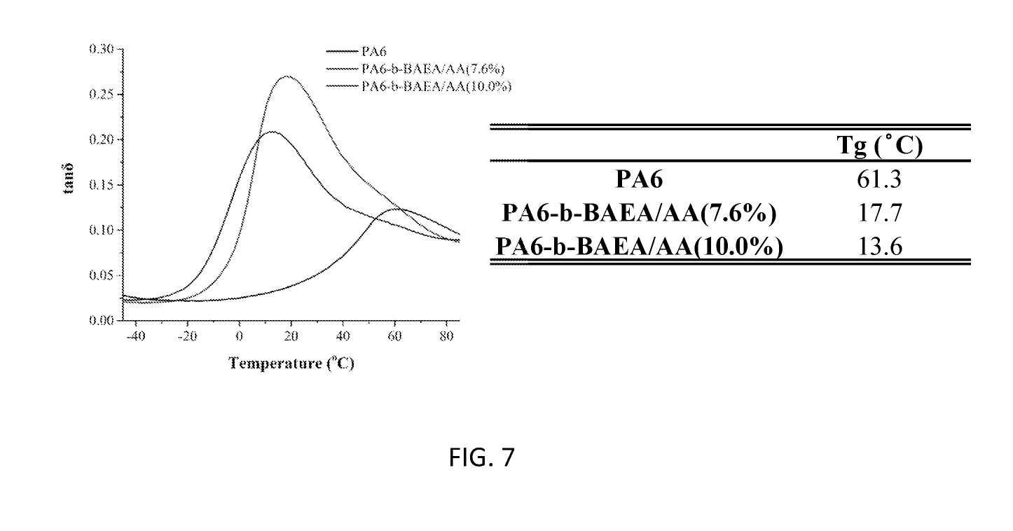

FIG. 5 shows FTIR spectra their analysis of the polyamide copolymers of examples 1 and 2 and the reference example 1 according to one embodiment of the invention. FIG. 6 shows DSC results of the polyamide copolymers of examples 1 and 2 and the reference example 1 according to one embodiment of the invention. FIG. 7 shows DMA results and glass transition temperatures of the polyamide copolymers of examples 1 and 2 and the reference example 1 according to one embodiment of the invention.

The mechanical properties of the polyamine copolymers of Examples 1 and 2 and the polyamines (PA6) of the reference example are shown in Table 2. As shown in Table 2, accompanying with the addition of BAEA/AA, the peel strength increased from 39.42 N/2.5 cm to 59.95 N/2.5 cm. As the copolymerization ratio is increased by 2.4 mole % (from 7.6% to 10.0%), the peel strength is increased by 52.1%. It is noted that the addition of BAEA/AA can effectively increase the bonding strength of the hot melt adhesive.

TABLE 2
The mechanical properties of PA6 and polyamide copolymers

| | Reference | Example 1 | Example 2 |
|---|---|---|---|
| | (PA6) | (7.6%) | (10.0%) |
| Tensile Strength | 79.73 ± 0.34 | 60.65 ± 4.80 | 48.51 ± 1.95 |
| (MPa) | | | |
| Breaking Strength | 60.88 ± 4.46 | 60.65 ± 4.80 | 48.51 ± 1.95 |
| (MPa) | | | |
| Elongation at break | 112.57 ± 32.85 | 14.93 ± 0.73 | 13.18 ± 0.70 |
| (%) | | | |
| T-peel Strength | — | 39.42 ± 6.38 | 59.95 ± 6.71 |
| (N/2.5 cm) | | | |

The moisture regains of the polyamine copolymers of Examples 1 and 2 and the polyamines (PA6) of the reference example are shown in Table 3. As shown in Table 3, accompanying with the addition of BAEA/AA, the moisture regain is increased.

TABLE 3
The moisture regains of PA6 and polyamide copolymers

Moisture regain

| | (20° C., RH = 65%) | (27° C., RH = 65%) |
|---|---|---|
| Reference (PA6) | 3.58 ± 0.42 | 5.48 ± 0.63 |
| Example 1 (7.6%) | 5.59 ± 0.18 | 9.53 ± 0.17 |
| Example 2 (10.0%) | 5.67 ± 0.28 | 9.66 ± 0.15 |

From the above results, it was found that the polyamide copolymer of the present invention produced by using a modifying agent of the present invention has a melting point, compared to the conventional nylon 6 (PA6) produced in the reference example. The low melting point is suitable as a low melting point nylon type hot melt adhesive and the copolymer also has the high T-peel strength. Examples 1 and 2 display that the copolymers have better hygroscopicity than that of PA6 when formed into a fiber. The reason is that BAEA contains EDA (Ethylene diamine) moieties which have higher hydrogen-bond density than that of existing diamine analogues. The advantage is such a high hydrogen-bond density which is still unable to be achieved by existing diamine analogues from other known technologies.

### EXAMPLE 3

(Synthesis of Amide- Group-Containing Diamine; BAEA)

BAEA was synthesized by the same method described in example 1.

(Synthesis of BAEA/IPA Salt)

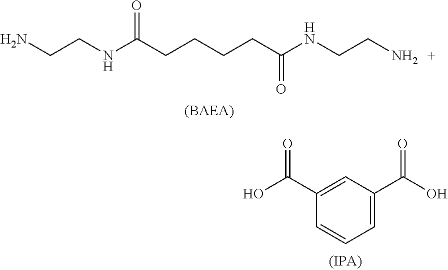 [SMILES: NCCNC(=O)CCCCC(=O)NCCN ; O=C(O)c1cccc(C(=O)O)c1]

(Synthesis of Polyamide Copolymer)

Except using BAEA/IPA salt instead of BAEA/AA salt in example 1, polyamide copolymer was synthesized using the method described in example 1.

### EXAMPLE 4

(Synthesis of Amide- Group-Containing Diamine; BAEA)

BAEA was synthesized by the same method described in example 1.

(Synthesis of BAEA/PTA Salt)

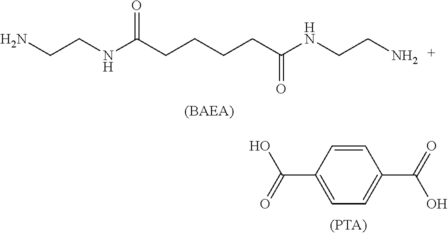 [SMILES: NCCNC(=O)CCCCC(=O)NCCN ; O=C(O)c1ccc(C(=O)O)cc1]

(Synthesis of Polyamide Copolymer)

Except using BAEA/PTA salt instead of BAEA/AA salt in example 1, polyamide copolymer was synthesized using the method described in example 1.

### EXAMPLE 5

(Synthesis of Amide- Group-Containing Diamine; BAES) (x=8, y=2 in the General Formula (I))

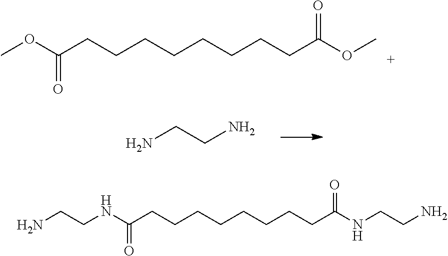

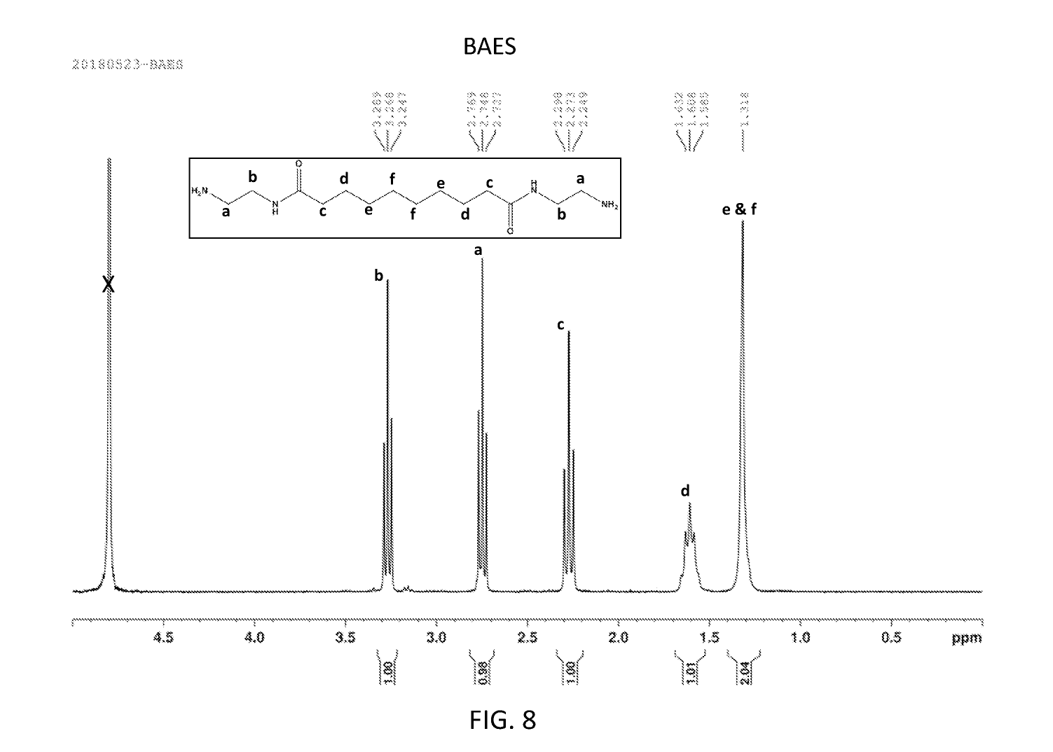

The BAES monomer was prepared using the same method as that of Example 1 in the above, except that the reactants added were dimethyl sebacate (1 mol, Dimethyl sebacate, DMS) and ethylenediamine (10 mole, Ethylenediamide, EDA) which were placed in a 1 L reaction flask, and methanol was added as a solvent. The copolymer product was obtained (Yelid=78.8%). The characteristic IR Spectral Absorption Peak (FTIR) was shown at 1665−1630 cm⁻¹ . (Carboxyl group of amide). FIG. 8 shows H-NMR spectrum of the modifying agent (BAES) according to another embodiment of the invention.

### EXAMPLE 6

(Synthesis of Amide- Group-Containing Diamine; BAHA) (x=4, y=6 in the General Formula (I))

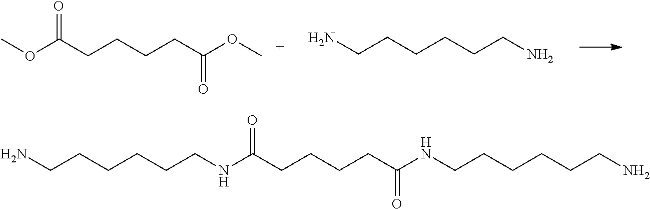

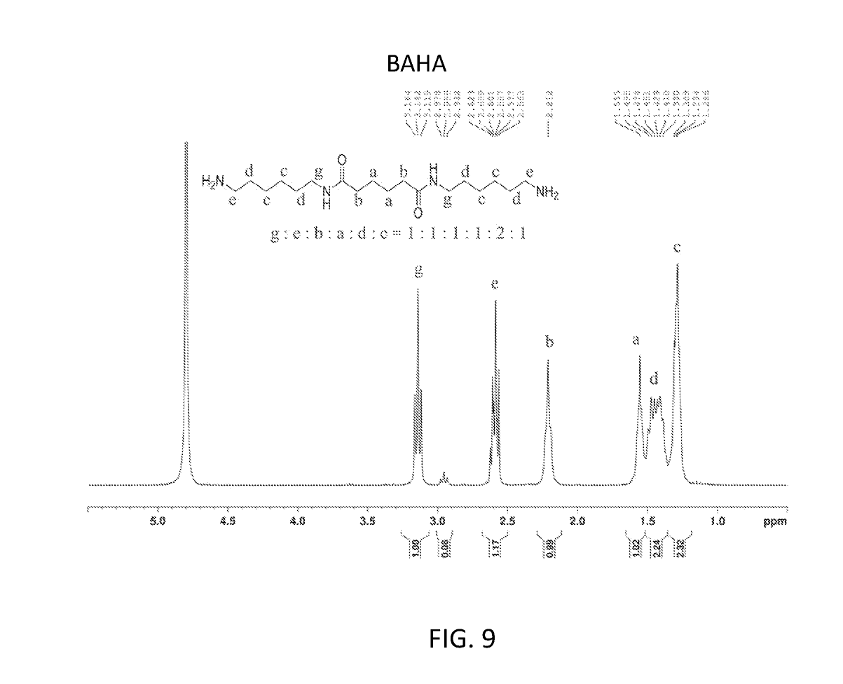

The BAHA monomer was prepared using the same method as that of Example 1 in the above, except that the reactants added were Dimethyl adipate (DMA) and hexamethylenediamine (10 mole, Hexamethylenediamine, HMDA) which were placed in a 1 L reaction flask, and methanol was added as a solvent. The copolymer product was obtained (Yelid=72%). The characteristic IR Spectral Absorption Peak (FTIR) was shown at 1665−1630 cm⁻¹(Carboxyl group of amide). FIG. 9 shows H-NMR spectrum of the modifying agent (BAHA) according to another embodiment of the invention.

### EXAMPLE 7

(Synthesis of BAHA/IPA Salt)

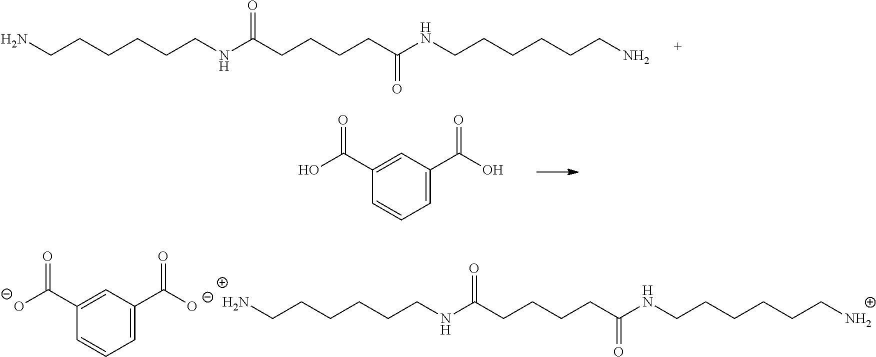 [SMILES: NCCCCCCNC(=O)CCCCC(=O)NCCCCCCN ; O=C(O)c1cccc(C(=O)O)c1 ; O=C([O-])c1cccc(C(=O)[O-])c1 ; [NH3+]CCCCCCNC(=O)CCCCC(=O)NCCCCCC[NH3+]]

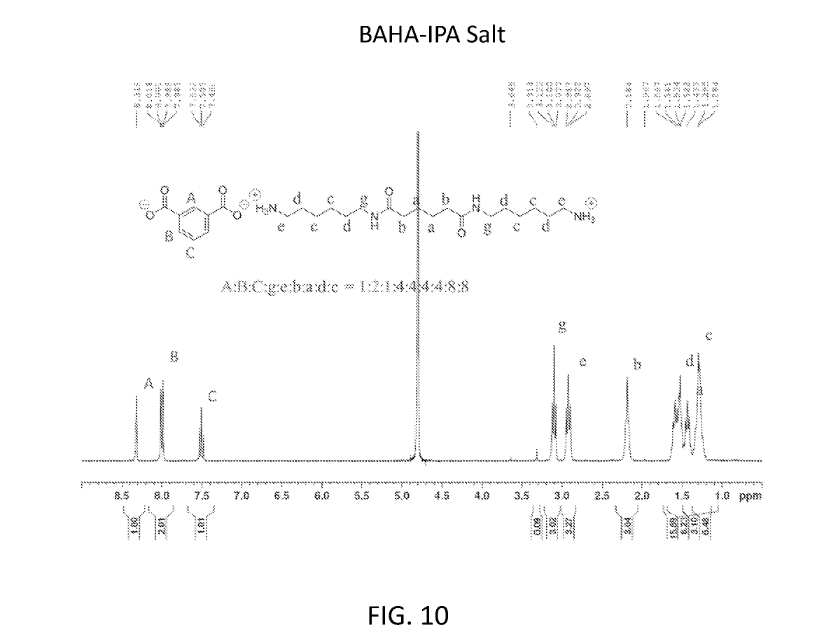

BAHA/IPA salt was synthesized using the same method as that of the Example 3, except using BAHA instead of BAEA. FIG. 10 shows H-NMR spectrum of the modifying agent (BAHA/IPA salt) according to another embodiment of the invention.

In conclusion, according to the present invention, a modifying agent, a polyamide copolymer synthesized by reacting the modifying agent with caprolactam, and the method for preparing a polyamide copolymer are provided. The obtained polyamide copolymer has a low melting temperature and a higher moisture regain than the traditional nylon 6 homopolymer. In addition, the obtained polyamide copolymer has a high T-peel strength suitable to be used as a low-melt adhesive. On the other hand, the method for preparing a polyamide copolymer according to the present invention has the advantages of using low cost raw materials and having easy processing conditions.

The foregoing description of the preferred embodiments of the invention has been presented for purposes of illustration and description. It is not intended to be exhaustive or to limit the invention to the precise form or to exemplary embodiments disclosed. Accordingly, the foregoing description should be regarded as illustrative rather than restrictive. Obviously, many modifications and variations will be apparent to practitioners skilled in this art. The embodiments are chosen and described in order to best explain the principles of the invention and its best mode practical application, thereby to enable persons skilled in the art to understand the invention for various embodiments and with various modifications as are suited to the particular use or implementation contemplated. It is intended that the scope of the invention be defined by the claims appended hereto and their equivalents in which all terms are meant in their broadest reasonable sense unless otherwise indicated. Therefore, the term “the invention”, “the present invention” or the like does not necessarily limit the claim scope to a specific embodiment, and the reference to particularly preferred exemplary embodiments of the invention does not imply a limitation on the invention, and no such limitation is to be inferred. The invention is limited only by the spirit and scope of the appended claims. The abstract of the disclosure is provided to comply with the rules requiring an abstract, which will allow a searcher to quickly ascertain the subject matter of the technical disclosure of any patent issued from this disclosure. It is submitted with the understanding that it will not be used to interpret or limit the scope or meaning of the claims. Any advantages and benefits described may not apply to all embodiments of the invention. It should be appreciated that variations may be made in the embodiments described by persons skilled in the art without departing from the scope of the invention as defined by the following claims. Moreover, no element and component in the present disclosure is intended to be dedicated to the public regardless of whether the element or component is explicitly recited in the following claims. Each of the terms “first” and “second” is only a nomenclature used to modify its corresponding element. These terms are not used to set up the upper limit or lower limit of the number of elements.

What is claimed is:

1. A modifying agent, for synthesizing a polyamine copolymer, being a amide- group-containing diamine which is a reaction product of an aliphatic diamine and a first carboxylate, or a salt formed by reacting the amide- group-containing diamine with a second carboxylic acid, wherein the amide- group-containing diamine is represented by the following general formula (I): 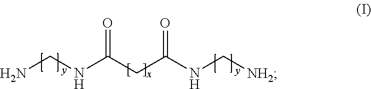
where y represents an integer within 2˜6 (2≤y≤6) and x represents an integer within 4˜18 (4≤x≤18);
the first carboxylate is an ester formed from an aliphatic carboxylic acid and the second carboxylic acid is a carboxylic acid.

2. The modifying agent as claimed in claim 1, wherein y is 2 and x is an integer within 4˜18.

3. The modifying agent as claimed in claim 1, wherein the amide- group-containing diamine has a boiling point higher than 180° C.

4. The modifying agent as claimed in claim 1, being a diamine dicarboxylate salt which is a reaction product of the amide- group-containing diamine and the second carboxylic acid selected from the group consisting of the following or the combination thereof: isophthalic acid, terephthalic acid and adipic acid.

5. The modifying agent as claimed in claim 1, wherein the polyamide copolymer synthesized by reacting the modifying agent and caprolactam has a moisture regain more than 5% at 20° C. and 65% RH.

6. The modifying agent as claimed in claim 1, wherein the aliphatic diamine is ethylene diamine and the first carboxylate is one ester selected from the group consisting of the following or the combination thereof: succinate, adipate, suberate, sebacate, and dodecanedioate.

7. A polyamide copolymer, being synthesized by reacting a modifying agent with caprolactam, wherein the polyamide copolymer has a lower melting temperature than polycaprolactam homopolymer; and
the modifying agent is an amide- group-containing diamine which is a reaction product of an aliphatic diamine and a first carboxylate, or a salt formed by reacting the amide- group-containing diamine with a second carboxylic acid, wherein the amide- group-containing diamine is represented by the following general formula (I): 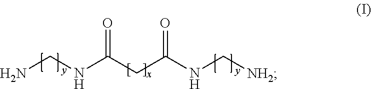
where y represents an integer within 2˜6 (2≤y≤6) and x represents an integer within 4˜18 (4≤x≤18);
the first carboxylate is an ester formed from an aliphatic carboxylic acid and the second carboxylic acid is a carboxylic acid.

8. The polyamide copolymer as claimed in claim 7, wherein the modifying agent is a diamine dicarboxylate salt which is a reaction product of the amide- group-containing diamine and the second carboxylic acid selected from the group consisting of the following or the combination thereof: isophthalic acid, terephthalic acid and adipic acid.

9. The polyamide copolymer as claimed in claim 7, wherein y is 2 and x is an integer within 4˜18.

10. The polyamide copolymer as claimed in claim 7, wherein the aliphatic diamine is ethylene diamine and the first carboxylate is one ester selected from the group consisting of the following or the combination thereof: succinate, adipate, suberate, sebacate, and dodecanedioate.

11. The polyamide copolymer as claimed in claim 7, wherein the polyamide copolymer has a moisture regain more than 5% at 20° C. and 65% RH.

12. A method for preparing a polyamide copolymer, comprising the following steps:
a step of providing a modifying agent, being a amide- group-containing diamine which is a reaction product of an aliphatic diamine and a first carboxylate, or a salt formed by reacting the amide- group-containing diamine with a second carboxylic acid; and
a step of copolymerizing the modifying agent with caprolactam;
wherein the amide- group-containing diamine is represented by the following general formula (I): 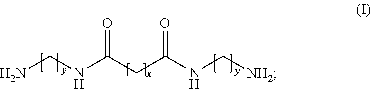
where y represents an integer within 2˜6 (2≤y≤6) and x represents an integer within 4˜18 (4≤x≤18);
the first carboxylate is an ester formed from an aliphatic carboxylic acid and the second carboxylic acid is a carboxylic acid.

13. The method as claimed in claim 12, wherein the step of copolymerizing is conducted under an atmospheric pressure of 1 atm.

14. The method as claimed in claim 12, wherein the second carboxylic acid is selected from the group consisting of the following or the combination thereof:
isophthalic acid, terephthalic acid and adipic acid.

15. The method as claimed in claim 12, wherein y is 2 and x is an integer within 4˜18.

16. The method as claimed in claim 12, wherein the aliphatic diamine is ethylene diamine and the first carboxylate is one ester selected from the group consisting of the following or the combination thereof: succinate, adipate, suberate, sebacate, and dodecanedioate.
